(19) United States
(12) Patent Application Publication
(10) Pub. No.: US 2026/0014868 A1
(43) Pub. Date: Jan. 15, 2026

(54) MOTION SICKNESS REDUCING APPARATUS AND METHOD FOR REDUCING MOTION SICKNESS

(71) Applicant: Stanley Electric Co., Ltd., Tokyo (JP)

(72) Inventors: Yuki ISHII, Meguro-ku, Tokyo (JP); Yuko MURAMATSU, Meguro-ku, Tokyo (JP); Keisuke MATSUBARA, Meguro-ku, Tokyo (JP); Kazuaki OE, Meguro-ku, Tokyo (JP); Naoya OSHIDA, Meguro-ku, Tokyo (JP)

(73) Assignee: Stanley Electric Co., Ltd., Tokyo (JP)

(21) Appl. No.: 18/996,259

(22) PCT Filed: Jul. 21, 2023
(86) PCT No.: PCT/JP2023/026888

§ 371 Date: Jan. 17, 2025

(30) Foreign Application Priority Data
Jul. 25, 2022 (JP) 2022-117739

(51) Int. Cl. B60K 35/29; B60K 35/22; B60K 35/28; G09G 3/20

(52) U.S. Cl. CPC B60K 35/29; B60K 35/22; B60K 35/28; G09G 3/2092; B60K 2360/182; B60K 2360/191; B60K 2360/332; B60K 2360/785; B60K 2360/794

(57) ABSTRACT

There is provided a motion sickness reducing apparatus capable of providing visual information to an individual on a vehicle as to how much he or she is tilted to the horizontal (as a result, motion sickness caused by sense of balance can be reduced.). The motion sickness reducing apparatus includes a display device that is provided in a riding space of a vehicle having the riding space in which an individual rides and displays a horizontal line extending in a horizontal direction orthogonal to a gravity direction of the vehicle in a riding space regardless of a tilt of the vehicle.

### TECHNICAL FIELD

The present disclosure relates to an apparatus and method for reducing motion sickness, and more particularly to an apparatus and method for reducing motion sickness, which can provide visual information to an individual on a vehicle as to how much he or she is inclined to the horizontal (as a result, motion sickness caused by sense of balance can be reduced.).

### BACKGROUND ART

As a countermeasure against motion sickness, an optical array system mounted on a vehicle and displaying a flow of light corresponding to the motion of the vehicle is known (see, for example, Patent Literature 1.).

### CITATION LIST

### Patent Literature

Patent Literature 1: Published Japanese Translation of PCT International Publication for Patent Application, No. 2019-523657

### SUMMARY OF INVENTION

### Technical Problem

However, upon examination by the inventors, it was found that in the optical array system described in Patent Document 1, the flow of light displayed mimics specific movements of the vehicle, and since there is no visual information for the individual riding in the vehicle about how much they are tilted relative to the horizontal, it is insufficient for addressing motion sickness caused by the sense of balance.

The present disclosure has been made in order to solve such problems, and it is an object of the present disclosure to provide a motion sickness reducing apparatus and a motion sickness reducing method capable of providing visual information to an individual on a vehicle as to how much he or she is inclined to the horizontal (as a result, motion sickness caused by sense of balance can be reduced.).

### Solution to Problem

A motion sickness reducing apparatus according to the present disclosure includes a display device that is provided in a riding space of a vehicle having the riding space in which an individual rides and displays a horizontal line extending in a horizontal direction orthogonal to a gravity direction of the vehicle in the riding space regardless of an inclination of the vehicle.

With such a configuration, it is possible to provide visual information to an individual on a vehicle as to how much he or she is inclined to the horizontal (as a result, motion sickness caused by sense of balance can be reduced.).

This is achieved by displaying a horizontal line extending in the horizontal direction orthogonal to the gravitational direction of the vehicle regardless of the inclination of the vehicle in the riding space of the vehicle provided with the riding space in which an individual rides.

Also, the above-described motion sickness reducing apparatus may further include a road condition detection unit for detecting a road condition where the vehicle is traveling; wherein the display device may display the horizontal line when the road condition satisfies a predetermined condition.

Also, in the above-described motion sickness reducing apparatus, the predetermined condition may be a slope, a curved road, or a straight road traveling at a predetermined acceleration force or less.

Also, in the above-described motion sickness reducing apparatus, the display device may display a bright spot moving along the horizontal line in a direction corresponding to an acceleration/deceleration of the vehicle together with the horizontal line.

Also, in the above-described motion sickness reducing apparatus, the display device may be provided on a windshield and a side glass constituting a wall surface surrounding the riding space.

Also, in the above-described motion sickness reducing apparatus, the display device may be further provided on a rear glass constituting the wall surface surrounding the riding space.

Also, in the above-described motion sickness reducing apparatus, the display device may be provided on a front dashboard and a side door provided in the riding space.

Also, in the above-described motion sickness reducing apparatus, the display device may be further provided on a rear dashboard provided in the riding space.

Also, in the above-described motion sickness reducing apparatus, the display device may be a film-shaped film light source.

Also, in the above-described motion sickness reducing apparatus, the film light source is an organic EL or a film-shaped LED.

Also, the above-described motion sickness reducing apparatus may further include a horizontal line setting unit for setting at least one of a color and width of the horizontal line; wherein the display device displays the horizontal line in the color, and width set.

Also, in the above-described motion sickness reducing apparatus, the display device displays an image including the horizontal line.

Also, in the above-described motion sickness reducing apparatus, the display device is provided on at least a part of a wall surface surrounding four sides of the riding space.

A method for reducing motion sickness according to the present disclosure is a method for displaying a horizontal line extending in a horizontal direction orthogonal to a gravity direction of a vehicle in a riding space of the vehicle provided with the riding space in which an individual rides regardless of an inclination of the vehicle.

With such the configuration, it is possible to provide visual information to an individual on a vehicle as to how much he or she is inclined to the horizontal (as a result, motion sickness caused by sense of balance can be reduced.).

This is achieved by displaying a horizontal line extending in the horizontal direction orthogonal to the gravitational direction of the vehicle regardless of the inclination of the vehicle in the riding space of the vehicle provided with the riding space in which an individual rides.

### Advantageous Effects of Invention

According to the present disclosure, it is possible to provide a motion sickness reducing apparatus and a motion sickness reducing method capable of providing visual information to an individual on a vehicle as to how much he or she is inclined to the horizontal (as a result, motion sickness caused by sense of balance can be reduced.).

### BRIEF DESCRIPTION OF THE DRAWINGS

FIG. 1 is a schematic view of a motion sickness reducing apparatus 10 (a film light source 20) provided in a riding space of a vehicle V;

FIG. 2A is an example of the front horizontal line HL_{20F} displayed on the front film light source 20F when the vehicle V is not inclined (for example, when the vehicle V is on a horizontal plane);

FIG. 2B is an example of the front horizontal line HL_{20F} displayed on the front film light source 20F when the vehicle V is tilted to the left (for example, when driving on a left curve);

FIG. 2C is an example of the front horizontal line HL_{20F} displayed on the front film light source 20F when the vehicle V is tilted to the right (for example, when driving on a right curve);

FIG. 3A is an example of the left horizontal line HL_{20L} displayed on the left film light source 20L when the vehicle V is not tilted (for example, when vehicle V is on a horizontal plane);

FIG. 3B is an example of the left horizontal line HL_{20L} displayed on the left film light source 20L when the vehicle V is tilted backward (for example, when driving uphill);

FIG. 3C is an example of the left horizontal line HL_{20L} displayed on the left film light source 20L when the vehicle V is tilted forward (for example, when driving downhill,);

FIG. 4 is a diagram for explaining the experimental environment performed by the present inventors;

FIG. 5 is a graph summarizing the results of the questionnaire conducted in the experiments performed by the present inventors;

FIG. 6 is another graph summarizing the results of the questionnaire conducted in the experiments performed by the present inventors;

FIG. 7 is a configuration diagram of the motion sickness reducing apparatus 10.

FIG. 8 is a diagram for explaining the X-axis and Y-axis of the inertial sensor 40 mounted on the vehicle V;

FIG. 9 is a view for explaining the outer shape of the film light source 20;

FIG. 10 is a flowchart of the horizontal line display processing 1;

FIG. 11A show an example of the horizontal line HL displayed as a result of the horizontal line display processing 1 (and the horizontal line display processing 2);

FIG. 11B show an example of the horizontal line HL displayed as a result of the horizontal line display processing 1;

FIG. 11C show an example of the horizontal line HL displayed as a result of the horizontal line display processing 2;

FIG. 12 is a flowchart of the horizontal line display processing 2;

FIG. 13 is a flowchart of the bright spot display processing;

FIG. 14A is example of bright spot display;

FIG. 14B is example of bright spot display;

FIG. 15A is a modified example of a partner provided with the film light source 20;

FIG. 15B is a modified example of a partner provided with the film light source 20;

FIG. 16 is a diagram showing the configuration of the motion sickness reducing apparatus 10 (variant example);

FIG. 17A is variation of the display form of the horizontal line HL;

FIG. 17B is variation of the display form of the horizontal line HL;

FIG. 18 shows a modification in which the liquid crystal panel 20A is used instead of the film light source 20; and

FIG. 19 shows a modification using a vehicle capable of automatic operation as the vehicle V.

### DESCRIPTION OF EMBODIMENTS

Hereinafter, a motion sickness reducing apparatus 10 according to an embodiment of the present disclosure will be described with reference to the accompanying drawings. In the respective drawings, corresponding components will be denoted by the same reference signs and repetitive descriptions will be omitted.

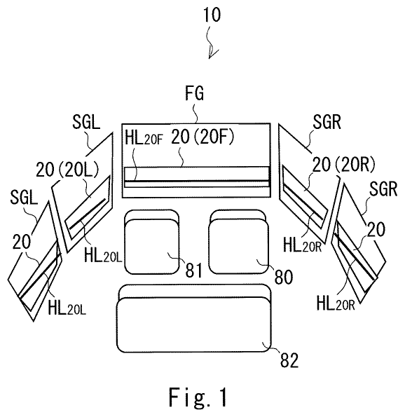

FIG. 1 is a schematic view of a motion sickness reducing apparatus 10 (a film light source 20) provided in a riding space of a vehicle V.

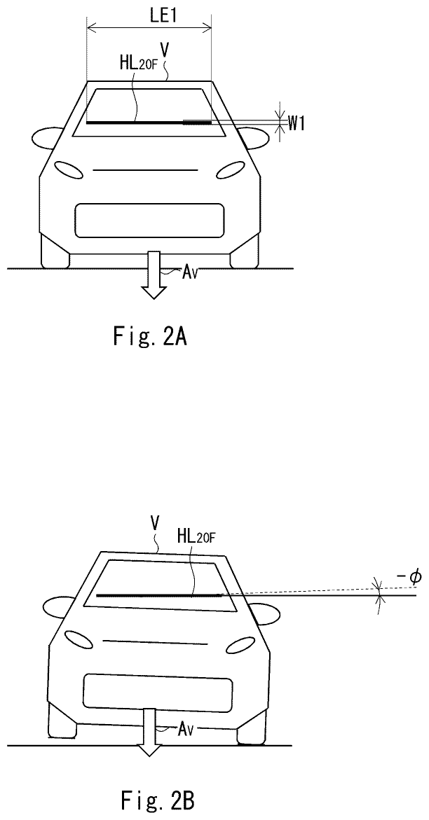

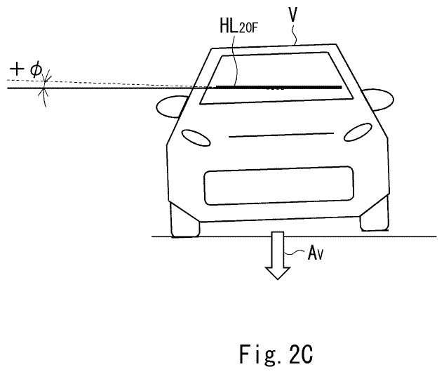

As shown in FIG. 1, the motion sickness reducing apparatus 10 is provided in a riding space of the vehicle V (see, for example, FIG. 2) having the riding space in which one or more individuals ride in order to reduce motion sickness. The motion sickness reducing apparatus 10 is provided with a film light source 20 (an example of a display device of the present disclosure) for displaying horizontal lines HL_{20F}, HL_{20L} and HL_{20R} (see FIG. 1) extending in a horizontal direction orthogonal to a gravity direction of the vehicle V in the riding space regardless of an inclination of the vehicle V. The vehicle V is, for example, an automobile. The film light source 20 is, for example, an organic EL or OP film. In FIG. 1, reference numeral 80 denotes a driver's seat, reference numeral 81 denotes a passenger's seat, and reference numeral 82 denotes a rear seat.

The film light source 20 is provided, for example, on a windshield FG, and side glasses SGL, SGR of the vehicle V. Hereinafter, the film light source 20 provided on the windshield FG is referred to as a front film light source 20F. Similarly, the film light source provided on a left side-glass SGL is referred to as a left film light source 20L. Similarly, the film light source provided on a right side-glass SGR is referred to as a right film light source 20R. If these are not specifically distinguished, they are simply referred to as the film light source 20.

The horizontal line HL displayed on the front film light source 20F is called a front horizontal line HL_{20F}. Similarly, the horizontal line HL displayed on the left film light source 20L is called a left horizontal line HL_{20L}. Similarly, the horizontal line HL displayed on the right film light source 20R is called a right horizontal line HL_{20R}. When these are not specifically distinguished, they are simply referred to as the horizontal line HL.

FIG. 2A is an example of the front horizontal line HL_{20F} displayed on the front film light source 20F when the vehicle V is not tilted (for example, when the vehicle V is on a horizontal plane). FIG. 2B is an example of the front horizontal line HL_{20F} displayed on the front film light source 20F when the vehicle V is tilted to the left (for example, when driving on a left curve). FIG. 2C is an example of the front horizontal line HL_{20F} displayed on the front film light source 20F when the vehicle V is tilted to the right (for example, when driving on a right curve).

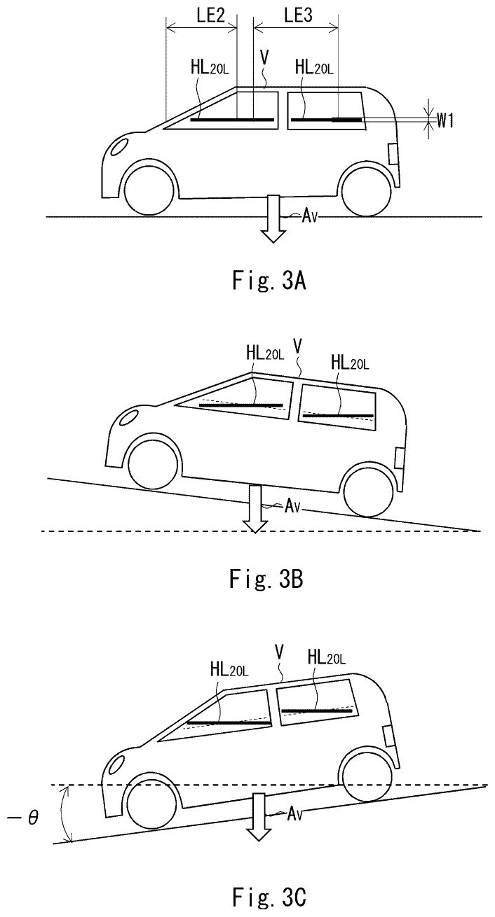

FIG. 3A is an example of the left horizontal line HL_{20L} displayed on the left film light source 20L when the vehicle V is not tilted (for example, when vehicle V is on a horizontal plane). FIG. 3B is an example of the left horizontal line HL_{20L} displayed on the left film light source 20L when the vehicle V is tilted backward (for example, when driving uphill). FIG. 3C is an example of the left horizontal line HL_{20L} displayed on the left film light source 20L when the vehicle V is tilted forward (for example, when driving downhill,). Although not shown, the right horizontal line HL_{20R} is also displayed on the right film light source 20R in the same manner as the left horizontal line HL_{20L}.

As shown in FIGS. 2A-2C and 3A-3C, the horizontal line HL (the front horizontal line HL_{20F}, the left horizontal line HL_{20L}, the right horizontal line HL_{20R}) is a light line (a line formed by light) displayed on the film light source 20 (the front film light source 20F, the left film light source 20L, the right film light source 20R) in a state extending in the horizontal direction orthogonal to the gravity direction A_{V} of the vehicle V regardless of the tilt of the vehicle V.

The present inventors confirmed through experiments that motion sickness can be reduced by displaying the horizontal line HL in the riding space of the vehicle V as described above.

The experiments performed by the present inventors will be described below.

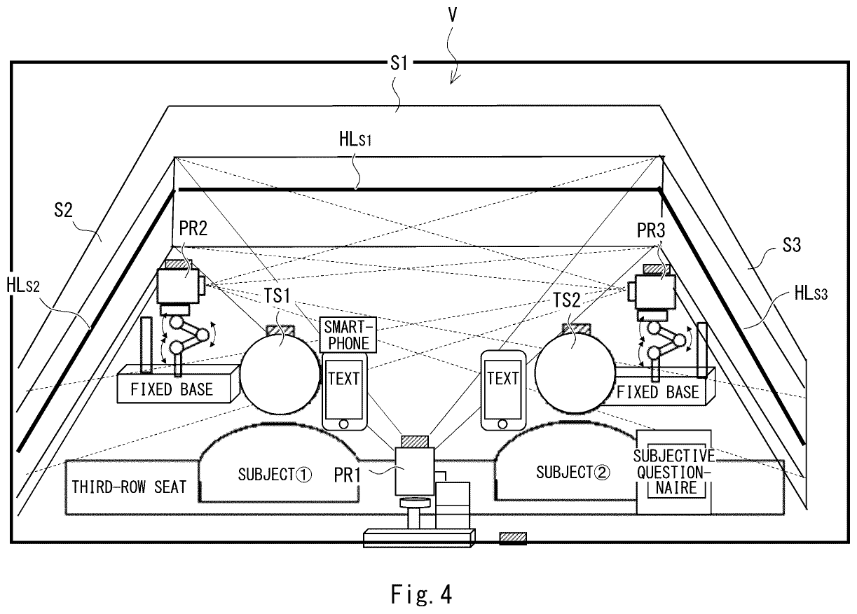

FIG. 4 is a diagram for explaining the experimental environment performed by the present inventors.

This experiment was conducted using an automobile as vehicle V. There were 45 subjects. For each subject, as shown in FIG. 4, this experiment was conducted twice in total, in an environment in which green horizontal lines HL_{S1}, HL_{S2}, and HL_{S3} were projected by projectors PR1 to PR3 on white screens S1 to S3 (screens that block light from the riding space of the rear seat) provided on the front and left and right sides of subject TS (in FIG. 4, TS1, TS2) seated in the rear seat of the automobile, and in an environment in which the horizontal lines HL_{S1}, HL_{S2}, and HL_{S3} were not projected. The width of the horizontal lines HL_{S1}, HL_{S2}, and HL_{S3} was about 1 cm. Hereinafter, the experiment conducted in the environment in which the horizontal lines HL_{S1}, HL_{S2}, and HL_{S3} were projected is called “experiment with horizontal lines”, and the experiment conducted in the environment in which the horizontal lines HL_{S1}, HL_{S2}, and HL_{S3} were not projected is called “experiment without horizontal lines”.

In order to maintain the level of the horizontal lines HL_{S1}, HL_{S2}, and HL_{S3}, that is, to make the horizontal lines HL_{S1}, HL_{S2}, and HL_{S3} extend in the horizontal direction orthogonal to the direction of gravity of the vehicle regardless of the tilt of the vehicle, the projectors PR1 to PR3 were mounted on stabilizers installed inside the automobile.

In both the experiment with horizontal lines and without the experiment without horizontal lines, the automobile was driven at 40 km/h on the same road (3 laps around a road with a total length of about 5 km, 30 curves, and a height difference of about 40 m).

In the experiments with the horizontal lines and the experiment without horizontal lines, each subject sat in the rear seat of the automobile traveling under the above conditions, read a text displayed on a mobile terminal (a smartphone or a tablet) in his/her hand, and input a response (self-assessment of the state of motion sickness rated on a 11-point scale from 0 to 10 [subjective evaluation]) to a questionnaire displayed on the mobile terminal every 2 minutes. In the experiment with a horizontal line, it is intended that a horizontal is recognized by “light” to a peripheral visual field of each subject, that is, horizontal lines HL_{S1}, HL_{S2}, and HL_{S3} displayed (projected) on the screens S1 to S3.

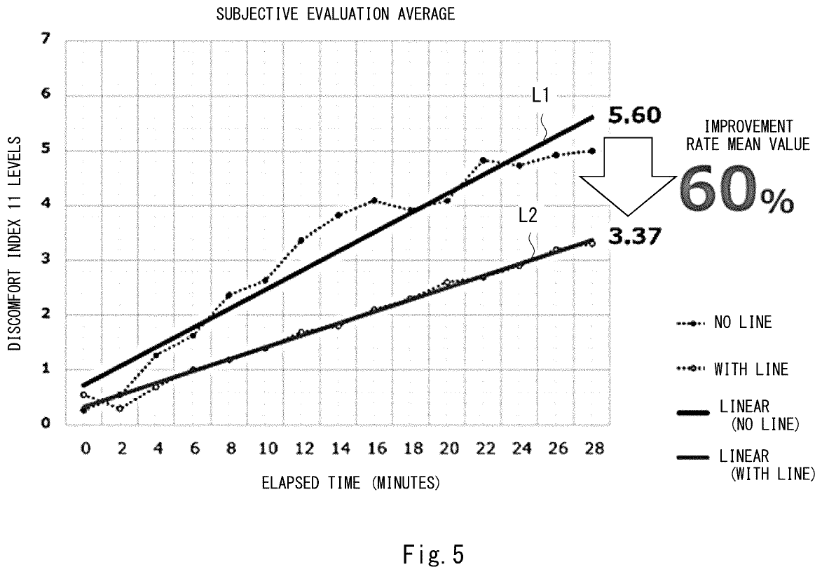

FIG. 5 is a graph summarizing the results of the questionnaire conducted in the experiments performed by the present inventors.

In FIG. 5, the vertical axis represents the discomfort index, which is a 11-point scale from 0 to 10 [subjective evaluation], while the horizontal axis represents the elapsed time (minutes) since the start of the experiment. In FIG. 5, the meanings of the black circle (plural), white circle (plural), lines L1 and L2, and the values “5.60” and “3.37” are as follows.

That is, the black circle (plural) represents the discomfort index (mean value) in the experiment without horizontal lines, that is, the mean value of the 11-point scale (subjective evaluation) entered by each subject in the experiment without horizontal lines. The line L1 represents an approximate line to the black circle (plural). On the other hand, the white circle (plural) represents the discomfort index (mean value) in the experiment with horizontal lines, that is, the mean value of the 11-point scale (subjective evaluation) entered by each subject in the experiment with horizontal lines. The line L2 represents an approximate line to the white circle (plural).

The value “5.60” represents the average value of the 11-point scale (discomfort index) entered last by each subject (after 28 minutes) in the experiment without the horizontal lines. On the other hand, the value “3.37” represents the average value of the 11-point scale (discomfort index) entered last by each subject (after 28 minutes) in the experiment with the horizontal lines.

Referring to FIG. 5, it can be seen that the discomfort index (mean value) decreased from 5.60 to 3.37 in the experiment with the horizontal line compared to the experiment without the horizontal line, that is, the improvement rate (mean value) was calculated as (3.37/5.60)×100, approximately 60%. The reason why “5.60” and “3.37” on the 11-point scale (discomfort index) entered last by each subject (after 28 minutes) were used is as follows. In other words, since the state of motion sickness (discomfort level) gradually increases over time, “5.60” and “3.37” on the 11-point scale (discomfort index) entered last by each subject (after 28 minutes) are considered to reflect the state of motion sickness (discomfort level) the most.

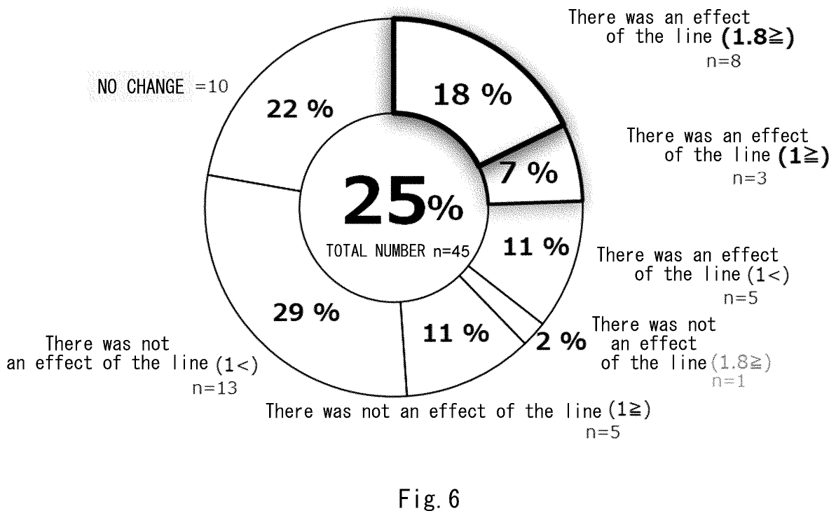

FIG. 6 is another graph summarizing the results of the questionnaire conducted in the experiments performed by the present inventors.

The number “18%” and “There was an effect of the line. (1.8≥)” in FIG. 6 represent the percentage of subjects (8 subjects) in which the difference ΔA (discomfort index A1−discomfort index A2) between the 11-point scale (hereinafter referred to as discomfort index A1) input last (after 28 minutes) in the experiment without horizontal line and the 11-point scale (hereinafter referred to as discomfort index A2) input last (after 28 minutes) in the experiment with horizontal line was 1.8 or more. Similarly, the number “7%” and “There was an effect of the line. (1.8≥)” represent the percentage of subjects (3 subjects) in which the difference ΔA (discomfort index A1−discomfort index A2) was less than 1.8 and more than 1. Similarly, the number “11%” and “There was an effect of the line. (1<)” represent the percentage of subjects (5 subjects) in which the difference ΔA (discomfort index A1−discomfort index A2) was less than 1 and 0 or more. In FIG. 6, There was an effect=A1−A2=+1.8 is described as “There was an effect of the line. (1.8≥)”, while there was not an effect=A1−A2=−1.8 is described as “There was not an effect of the line. (1.8≥)”.

Here, when the difference ΔA (discomfort index A1−discomfort index A2) is positive, it indicates that the discomfort index was reduced by displaying (projecting) the horizontal lines HL. In other words, when the difference ΔA (discomfort index A1−discomfort index A2) is positive, it indicates that motion sickness was reduced by displaying (projecting) the horizontal lines HL.

Referring to FIG. 6, it can be seen that the subjects whose difference ΔA (discomfort index A1−discomfort index A2) is positive, that is, the subjects whose motion sickness was reduced by displaying (projecting) the horizontal lines HL, are 16 subjects (about 36% of the total subjects): subjects (8 subjects) whose difference ΔA is 1.8 or more, subjects (3 subjects) whose difference ΔA is less than 1.8 and more than 1, and subjects (5 subjects) whose difference ΔA is less than 1 and more than 0.

From the above experiments, it can be seen that motion sickness can be reduced by displaying the horizontal lines HL in the riding space (for example, film light source 20 provided in the riding space) of the vehicle V.

The reason why motion sickness can be reduced by displaying the horizontal lines HL in the riding space of the vehicle V can be considered as follows.

First, the cause of motion sickness is generally considered as follows. That is, when a vehicle (for example, an automobile) causes irregular acceleration and deceleration, repeated stops and departures, and driving on roads with continuous ups and downs and curves, back-and-forth, side-to-side, and up-and-down motions occur. This creates a mismatch between the information transmitted to the brain via vision and the information (such as body position, sway, and speed) transmitted to the brain from the inner ear (semicircular canals and otolith organs). As a result, the brain is unable to process the information, leading to the symptoms of motion sickness. (https://www.ssp.co.jp/aneron/cause/mechanism.html)

On the other hand, as in the above experiment, by displaying the horizontal line HL in the riding space of the vehicle, it is possible to suppress (or reduce) the difference between the information input through vision and transmitted to the brain and the information (for example, body position, sway, speed) input from the inner ear (semicircular canals and otolith organs) and transmitted to the brain. As a result, motion sickness seems to have been reduced.

Next, a configuration example of the motion sickness reducing apparatus 10 will be described.

The motion sickness reducing apparatus 10 is mounted on a vehicle V. An example in which the vehicle V is an automobile will be described below.

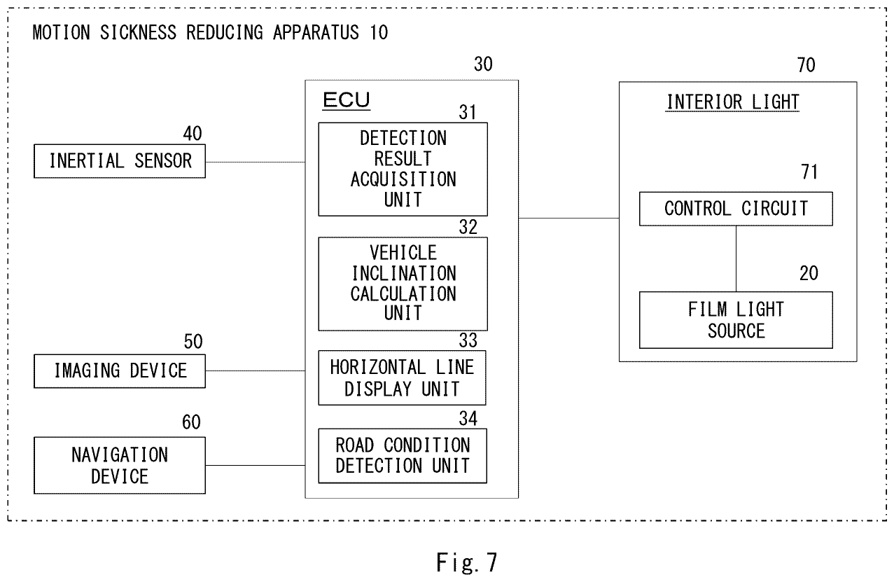

FIG. 7 is a configuration diagram of the motion sickness reducing apparatus 10.

As shown in FIG. 7, the motion sickness reducing apparatus 10 includes an ECU (Electronic Control Unit) 30, an inertial sensor 40, an imaging device 50, a navigation device 60, and an interior light 70.

The ECU30 is, for example, a control device including a CPU, a RAM, and a ROM, although not shown. The ECU30 functions as a detection result acquisition unit 31, a vehicle tilt calculation unit 32, a horizontal line display unit 33, and a road condition detection unit 34 when the CPU executes a predetermined program read from the ROM into the RAM. A part or all of these units may be realized by hardware.

The ECU30 is electrically connected to the inertial sensor 40, the imaging device 50, the navigation device 60, and the interior light 70.

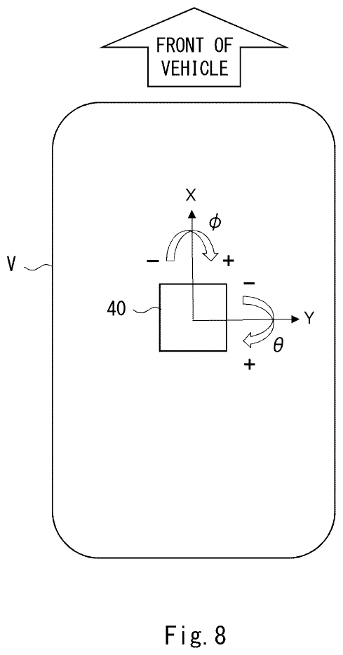

The inertial sensor 40 is a sensor for detecting an inertial force (Acceleration in X, Y, and Z axes, and angular velocity in X, Y, and Z axes) applied to the vehicle V. As shown in FIG. 8, the X-axis extends in the longitudinal direction of the vehicle V, the Y-axis extends in the width direction of the vehicle V, and the Z-axis extends in the direction orthogonal to the X-axis and the Y-axis. FIG. 8 is a diagram for explaining the X-axis and Y-axis of the inertial sensor 40 mounted on the vehicle V. As the inertial sensor 40, for example, a 6-axis IMU (Inertial Measurement Unit) or a 3-axis IMU can be used. The inertial sensor 40 may be one (reference) or multiple. The inertial sensor 40 is provided at a position (optimum position) which is considered so that the inertial force (acceleration in X, Y, and Z axes, and angular velocity in X, Y, and Z axes) applied to the vehicle V can be detected.

The imaging device 50 is a camera (including imaging elements such as CCD sensors and CMOS sensors) for taking an image of the front side of the vehicle V, and is provided at a predetermined position (for example, a riding space) of the vehicle V. An image (image data) taken by the imaging device 50 is input to the ECU30.

Although not shown, the navigation device 60 includes a route search unit, a route guidance unit, a current position detection unit, a map information storage unit, and the like. The route search unit searches a route from a starting place (for example, the current position of the vehicle V) to a destination of the vehicle V based on map information stored in the map information storage unit. The route guidance unit guides the route searched by the route search unit. The current position detection unit detects the current position of the vehicle V based on a signal from a GPS (not shown) provided in the vehicle V. The map information storage unit stores map information.

The detection result acquisition unit 31 acquires the detection result (acceleration in X, Y, and Z axes, and angular velocity in X, Y, and Z axes) of the inertial sensor 40 from the inertial sensor 40.

The vehicle tilt calculation unit 32 calculates the tilt (roll angle φ, pitch angle θ, etc. See FIG. 8) of the vehicle V based on the detection result acquired by the detection result acquisition unit 31.

The horizontal line display unit 33 creates a horizontal line HL based on the tilt (roll angle φ, pitch angle θ, etc.) of the vehicle V calculated by the vehicle tilt calculation unit 32, and controls a control circuit 71 to display the created horizontal line HL on the film light source 20. The width W1 (see FIG. 2A) and the length LE1 to LE3 (see FIG. 2A and 3A) of the horizontal line HL may be of an appropriate width and length. The color and brightness (luminance) of the horizontal line HL may be of an appropriate color and brightness. The color of the horizontal line HL may be a single color or a plurality of colors.

The road condition detection unit 34 detects a road condition where the vehicle V is traveling (such as a road condition corresponding to a current position of the vehicle V) based on information input from the imaging device 50 and the navigation device 60.

The interior light 70 includes a control circuit 71 and the film light source 20.

The control circuit 71 controls the film light source 20 so that the horizontal line HL generated by the horizontal line display unit 33 is displayed on the film light source 20 according to the control from the ECU30.

The film light source 20 is a film-shaped film light source (display) for displaying the horizontal line HL and is provided in the riding space. The film light source 20 is an example of a display device according to the present disclosure. As the film light source 20, for example, an organic EL (including an organic LED) or a film-shaped LED can be used. The film-shaped LED is a film light source including a plurality of semiconductor light-emitting elements which are fixed to a flexible film in a two-dimensionally (or three-dimensionally) arrangement.

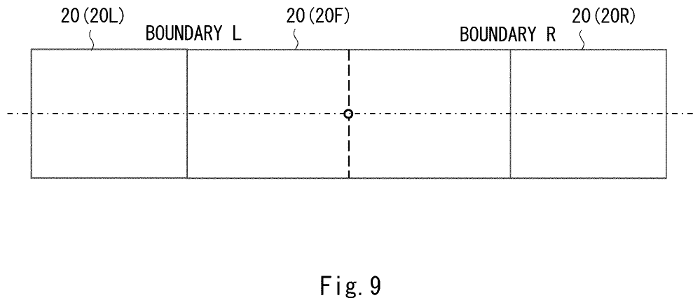

As shown in FIG. 1, the film light source 20 (front film light source 20F, left film light source 20L, right film light source 20R) is provided, for example, on the windshield FG, side glasses SGL, SGR of the vehicle V constituting a wall surface surrounding the riding space. As shown in FIG. 9, the outer shape of the film light source 20 is, for example, rectangular. FIG. 9 is a view for explaining the outer shape of the film light source 20. Reflective films (half mirror, white film, milky white film) may be laminated on the back surface of the film light source 20. A part of the windshield FG, side glasses SGL, SGR may be made milky white. The film light source 20 may also be provided on a rear glass (not shown) that forms the wall surface surrounding the riding space.

Next, a horizontal line display processing 1 will be described.

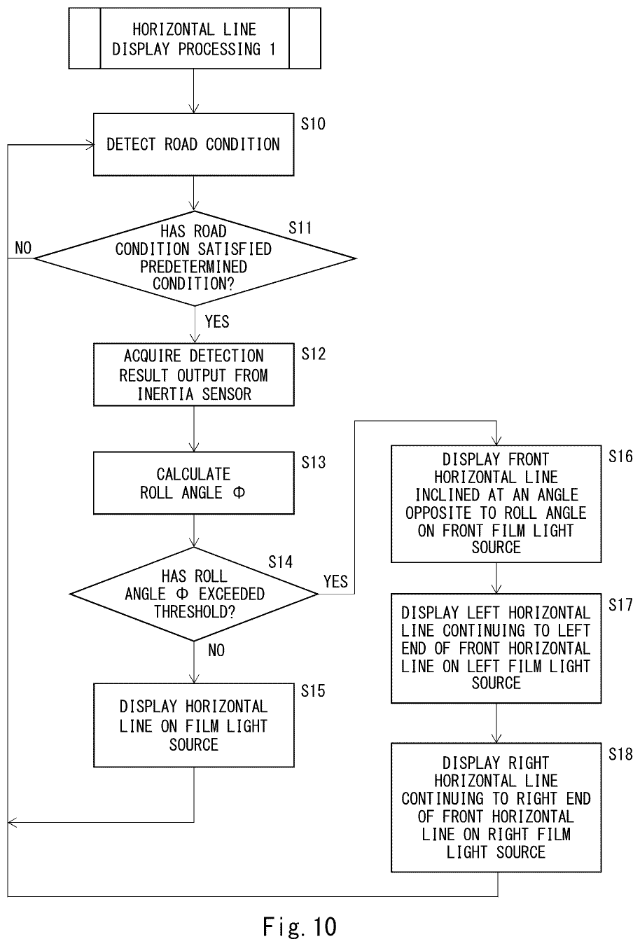

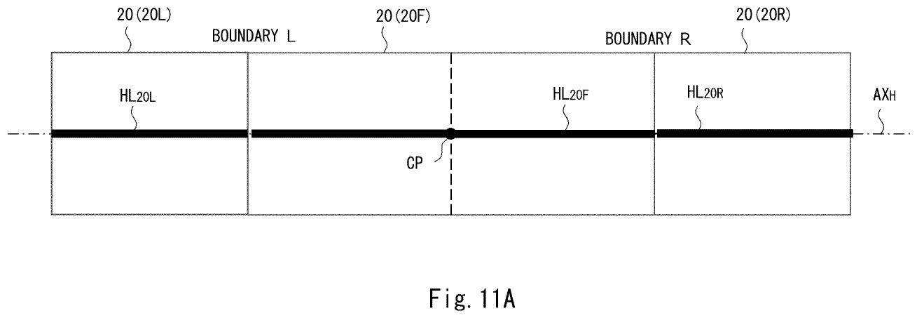

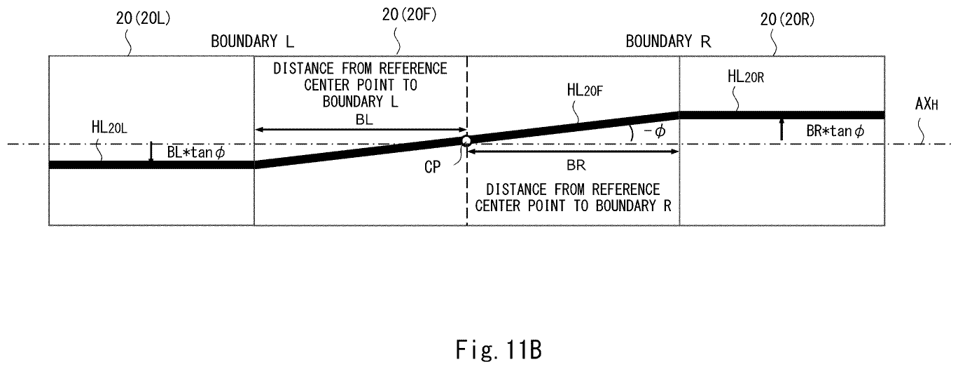

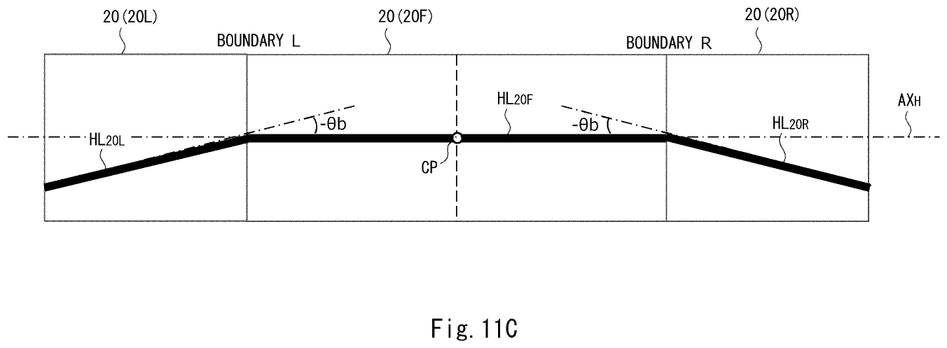

FIG. 10 is a flowchart of the horizontal line display processing 1. FIGS. 11A and 11B show an example of the horizontal line HL displayed as a result of the horizontal line display processing 1.

Hereinafter, as the horizontal line display processing 1, a process of displaying the horizontal line HL on the film light source 20 will be described when the vehicle V is tilted at an angle +φ (hereinafter, it is referred to as roll angle +φ. See FIG. 2C) to the right (right in direction toward front side of the vehicle). Hereinafter, in order to simplify the description, only the roll angle +φ will be considered, and the pitch angle θ will not be considered.

First, a road condition is detected (step S10). This is realized by the road condition detection unit 34.

Next, it is determined whether the road condition detected in step S10 satisfies a predetermined condition (step S11). This is realized by the ECU30 executing a predetermined program. The predetermined condition is a condition used for determining whether or not the road condition (the road currently being driven or the road to be driven) detected in step S10 is a road condition likely to induce motion sickness, and is, for example, a slope (a slope having a certain gradient or more), a curved road (a curved road having a certain curvature or more), or a straight road traveling at a predetermined acceleration force or less.

As a result, when the road condition detected in step S10 are determined to meet the predetermined condition (step S11: YES), the processes from step S12 onwards are executed.

Next, the detection result output from the inertial sensor 40 is acquired (step S12). This is realized by the detection result acquisition unit 31.

Next, the roll angle φ is calculated (step S13). This is realized by the vehicle tilt calculation unit 32. Here, it is assumed that the roll angle +φ was calculated by the vehicle V tilting (see FIG. 2C) to the right (right in direction toward front side of the vehicle).

Next, it is determined whether the roll angle φ exceeds a threshold value (step S14). This is realized by the ECU30 executing a predetermined program.

As a result, when it is determined that the roll angle +φ does not exceed the threshold (No in step S14), that is, when the vehicle V is hardly tilted to the left or right, the horizontal line HL is displayed on the film light source 20 (step S15). Specifically, as shown in FIG. 11A, the front horizontal line HL_{20F} passing through a reference center point CP and extending parallel to the reference horizontal line AX_{H} is displayed on the front film light source 20F. Similarly, the left horizontal line HL_{20L} passing through the reference center point CP and extending parallel to the reference horizontal line AX_{H} is displayed on the left film light source 20L. Similarly, the right horizontal line HL_{20R} passing through the reference center point CP and extending parallel to the reference horizontal line AX_{H} is displayed on the right film light source 20R.

On the other hand, when it is determined that the roll angle φ exceeds the threshold as a result of the determination in step S14 (step $14: Yes), that is, when the vehicle V is tilted to the right (or to the left) (see FIG. 2C), as shown in FIG. 11B, the front horizontal line HL_{20F} tilted at an angle −φ (see FIG. 11B) opposite to the roll angle +φ (see FIG. 2C) is displayed on the front film light source 20F (step S16).

Next, the left horizontal line HL_{20L} continuing to the left end of the front horizontal line HL_{20F} is displayed on the left film light source 20L (step S17). The left horizontal line HL_{20L} is corresponds to the left horizontal line HL_{20L} shown in FIG. 11A, shifted downward by BL×tan φ from the reference horizontal line AX_{H}. BL is a distance between the reference center point CP and a boundary L (the boundary between the left film light source 20L and the front film light source 20F).

Next, the right horizontal line HL_{20R} continuing to the right end of the front horizontal line HL_{20F} is displayed on the right film light source 20R (step S18). The right horizontal line HL_{20R} is corresponds to the right horizontal line HL_{20R} shown in FIG. 11A, shifted upward by BR×tan φ from the reference horizontal line AX_{H}. BR is a distance between the reference center point CP and a boundary R (the boundary between the right film light source 20R and the front film light source 20F).

The processes of steps S12 to S18 are repeatedly executed as long as the road condition detected in step S10 satisfies the predetermined condition (while the determination result in step S11 is YES).

Next, a horizontal line display processing 2 will be described.

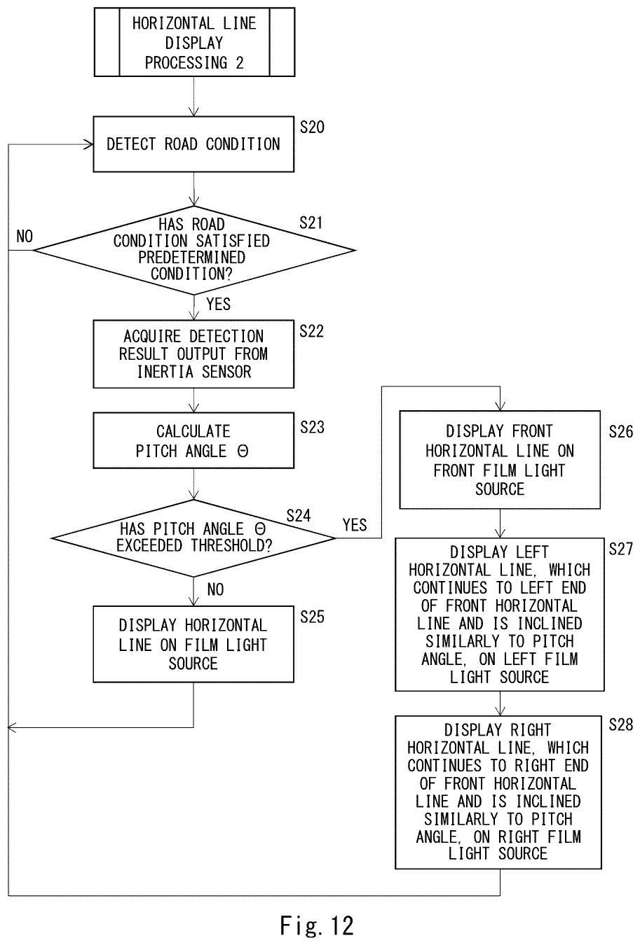

FIG. 12 is a flowchart of the horizontal line display processing 2. FIGS. 11A and 11B show an example of the horizontal line HL displayed as a result of the horizontal line display processing 2.

Hereinafter, as the horizontal line display processing 2, a process of displaying the horizontal line HL on the film light source 20 will be described when the vehicle V is tilted forward by an angle −θ (hereinafter, it is referred to as pitch angle −θ. See FIG. 3C). Hereinafter, in order to simplify the description, only the pitch angle −θ will be considered, and the roll angle φ will not be considered.

First, a road condition is detected (step S20). This is realized by the road condition detection unit 34.

Next, it is determined whether the road condition detected in step S20 satisfies a predetermined condition (step S21). This is realized by the ECU30 executing a predetermined program. The predetermined condition is a condition used for determining whether or not the road condition (the road currently being driven or the road to be driven) detected in step S20 is a road condition likely to induce motion sickness, and is, for example, a slope (a slope having a certain gradient or more), a curved road (a curved road having a certain curvature or more), or a straight road traveling at a predetermined acceleration force or less.

As a result, when the road condition detected in step S20 are determined to meet the predetermined condition (step S21: YES), the processes from step S22 onwards are executed.

Next, the detection result output from the inertial sensor 40 is acquired (step S22). This is realized by the detection result acquisition unit 31.

Next, the pitch angle θ is calculated (step S23). This is realized by the vehicle tilt calculation unit 32. Here, it is assumed that the pitch angle −θ was calculated by the vehicle V tilting forward (see FIG. 3C).

Next, it is determined whether the pitch angle θ exceeds a threshold value (step S24). This is realized by the ECU30 executing a predetermined program.

As a result, when it is determined that the pitch angle θ does not exceed the threshold (No in step S24), that is, when the vehicle V is hardly tilted forward or backward, the horizontal line HL is displayed on the film light source 20 (step S25). Specifically, as shown in FIG. 11A, the front horizontal line HL_{20F} passing through a reference center point CP and extending parallel to the reference horizontal line AX_{H} is displayed on the front film light source 20F. Similarly, the left horizontal line HL_{20L} passing through the reference center point CP and extending parallel to the reference horizontal line AX_{H} is displayed on the left film light source 20 L. Similarly, the right horizontal line HL_{20R} passing through the reference center point CP and extending parallel to the reference horizontal line AX_{H} is displayed on the right film light source 20R.

On the other hand, when it is determined that the pitch angle θ exceeds the threshold as a result of the determination in step S24 (step S24: Yes), that is, when the vehicle V is tilted forward (or backward), as shown in FIG. 11C, the front horizontal line HL_{20F} is displayed on the front film light source 20F (step S26). The front horizontal line HL_{20F} displayed here passes through the reference center point CP and extends parallel to the reference horizontal line AX_{H}.

Next, the left horizontal line HL_{20L} continuing to the left end of the front horizontal line HL_{20F} and tilted at an angle −θ (see FIG. 11C) similar to the pitch angle −θ (see FIG. 3C) is displayed on the left film light source 20L (step S27).

Next, the right horizontal line HL_{20R} continuing to the right end of the front horizontal line HL_{20F} and tilted at an angle −θ (see FIG. 11C) similar to the pitch angle −θ (see FIG. 3C) is displayed on the right film light source 20R (step S28).

The processes of steps S22 to S28 are repeatedly executed as long as the road condition detected in step S20 satisfies the predetermined condition (while the determination result in step S21 is YES).

Although the horizontal line display processing 1 and the horizontal line display processing 2 have been described separately, both processes may be executed simultaneously. Further, the following bright spot display processing may be executed in addition to these processes.

Next, the bright spot display processing will be described.

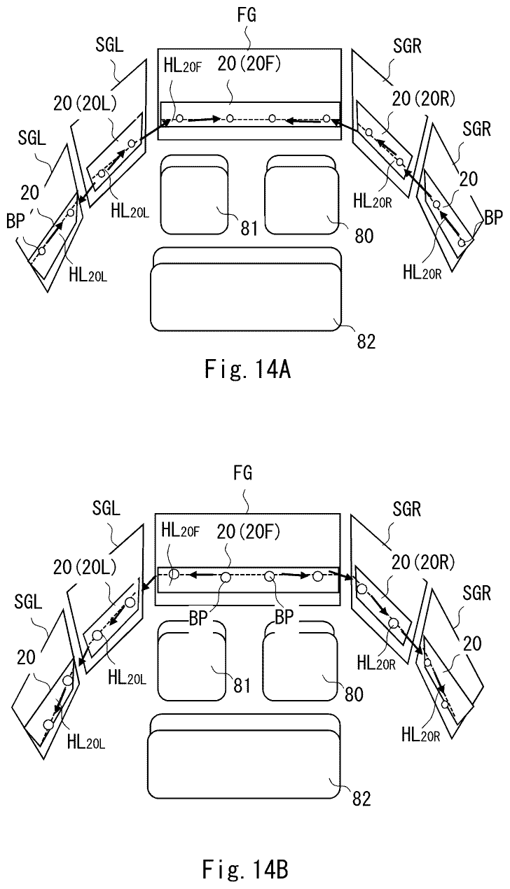

The bright spot display processing is a processing for displaying the bright spot BP (see FIGS. 14A and 14B) moving along the horizontal line HL in the direction corresponding to the acceleration/deceleration of the vehicle V together with the horizontal line HL displayed in the horizontal line display processing 1 or the horizontal line display processing 2.

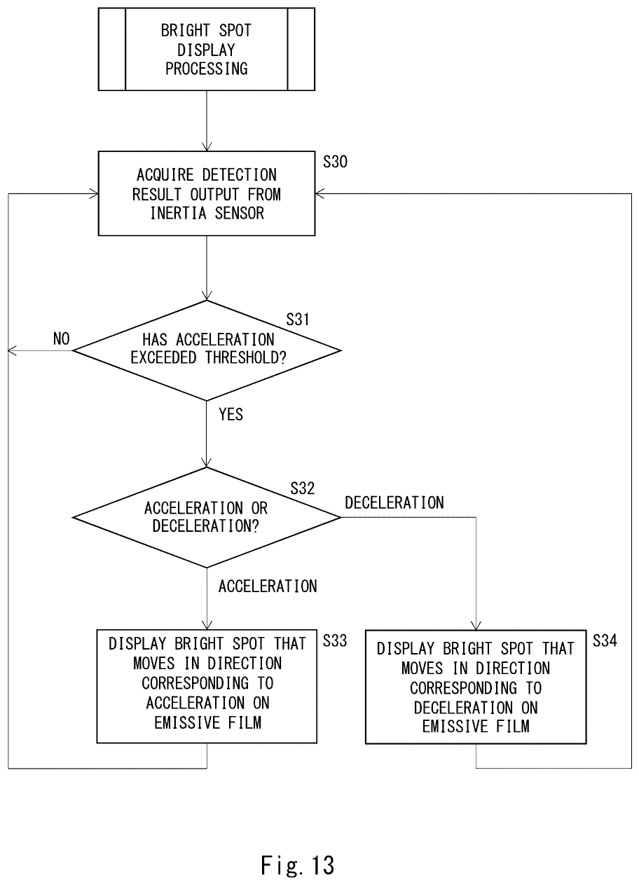

FIG. 13 is a flowchart of the bright spot display processing. FIGS. 14A and 14B are examples of bright spot display.

The bright spot display processing shown in FIG. 13 is executed, for example, after steps S25 and S28 shown in FIG. 12.

First, the detection result output from the inertial sensor 40 is acquired (step S30). This is realized by the detection result acquisition unit 31.

Next, it is determined whether the acceleration has exceeded the threshold (step S31), and when it is determined that the acceleration has exceeded the threshold, it is further determined whether the acceleration is an acceleration or a deceleration (step S32). This is realized by the ECU30 executing a predetermined program.

As a result, when it is determined that the acceleration is an acceleration (step S32: acceleration), as shown in FIG. 14A, the bright spot BP (see FIG. 14B) that moves along the horizontal line HL in a direction corresponding to the acceleration of the vehicle V, for example, in a direction starting from the rear end of the left film light source 20L and the right film light source 20R and toward the end of the center of the front film light source 20F (see the arrow in FIG. 14A) is displayed on the film light source 20, along with the horizontal line HL (step S15).

On the other hand, when it is determined that the acceleration is a deceleration (step S32: deceleration), as shown in FIG. 14B, the bright spot BP (see FIG. 14B) that moves along the horizontal line HL in a direction corresponding to the deceleration of the vehicle V, for example, in a direction starting from the center of the front film light source 20F and toward the end points of the rear ends of the left film light source 20L and the right film light source 20R (see the arrow in FIG. 14B) is displayed, along with the horizontal line HL.

The processes of steps S30 to S34 are repeatedly executed as long as the road condition detected in step S20 satisfies the predetermined condition (while the determination result in step S21 is YES).

In this way, by moving the bright spot BP so that it matches the bodily sensation, it is possible to provide information to the visual sense in the same way as the otoliths, which sense linear acceleration, and this is expected to reduce the motion sickness felt during acceleration and deceleration.

Conversely, when it is determined that the acceleration is an acceleration (step S32: acceleration), as shown in FIG. 14B, the bright spot BP (see FIG. 14B) that moves in a direction starting from the center of the front film light source 20F and toward the rear end of the left film light source 20L and the right film light source 20R (see the arrow in FIG. 14B) may be displayed, along with the horizontal line HL. Similarly, conversely, when it is determined that the acceleration is a deceleration (step S32: deceleration), as shown in FIG. 14A, the bright spot BP (see FIG. 14B) that moves in a direction starting from the end points of the rear ends of the left film light source 20L and the right film light source 20R and toward the center of the front film light source 20F (see the arrow in FIG. 14B) may be displayed on the film light source 20, along with the horizontal line HL. In this embodiment, the direction of movement of the bright spot is specified for acceleration and deceleration; however, depending on the occupants or road conditions, it may be preferable for the bright spot to move in the opposite direction.

As described above, according to the present embodiment, it is possible to give visual information of how much the individual is tilted to the horizontal to the individual who is on the vehicle V (as a result, motion sickness caused by sense of balance can be reduced.).

This is achieved by displaying a horizontal line HL extending in the horizontal direction orthogonal to the gravitational direction of the vehicle V regardless of the tilt of the vehicle V in the riding space of the vehicle V provided with the riding space in which an individual rides.

Additionally, by displaying the horizontal line HL, the individual riding in the vehicle V can predict the movement of the vehicle V, allowing them to naturally brace themselves for the vehicle's movement. It also provides a sense of security to the individual riding in the vehicle V.

Modified examples are described.

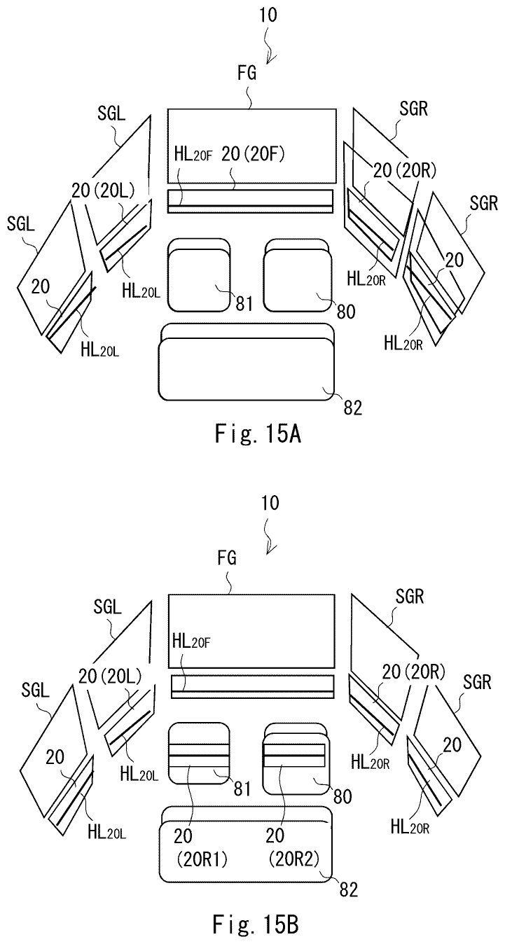

FIGS. 15A and 15B are variations of a portion where the film light source 20 is provided.

In the above embodiment, the film light source 20 is provided on the windshield FG, the side glasses SGL, SGR of the vehicle V constituting the wall surface surrounding the riding space (see FIG. 1), but this is not limited. For example, as shown in FIG. 15A, the film light source 20 may be provided on a front dashboard and a side door provided in the riding space. Further, as shown in FIG. 15B, the film light source 20 may be provided on a rear seat (for example, the rear side of the front seat, the rear dashboard).

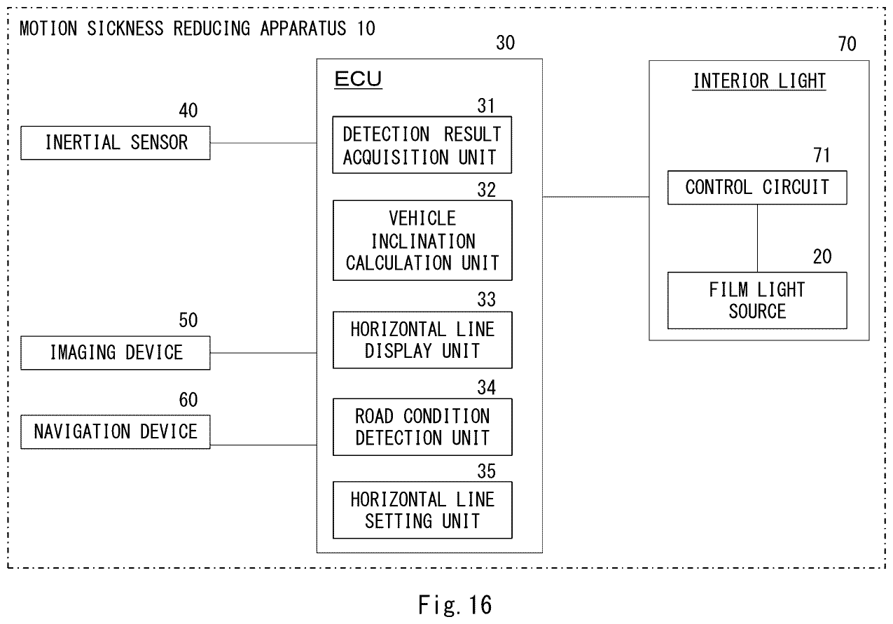

FIG. 16 is a diagram showing the configuration of the motion sickness reducing apparatus 10 (variant example).

As shown in FIG. 16, the motion sickness reducing apparatus 10 may further include a horizontal line setting unit 35. The horizontal line setting unit 35 sets at least one of the color and width of the horizontal line HL by user operation. Furthermore, the horizontal line setting unit 35 may set the brightness (luminance) of the horizontal line HL by user operation. Although not shown in the figures, the horizontal line HL is displayed on the film light source 20 in the color, width, and brightness (luminance) set by the horizontal line setting unit 35.

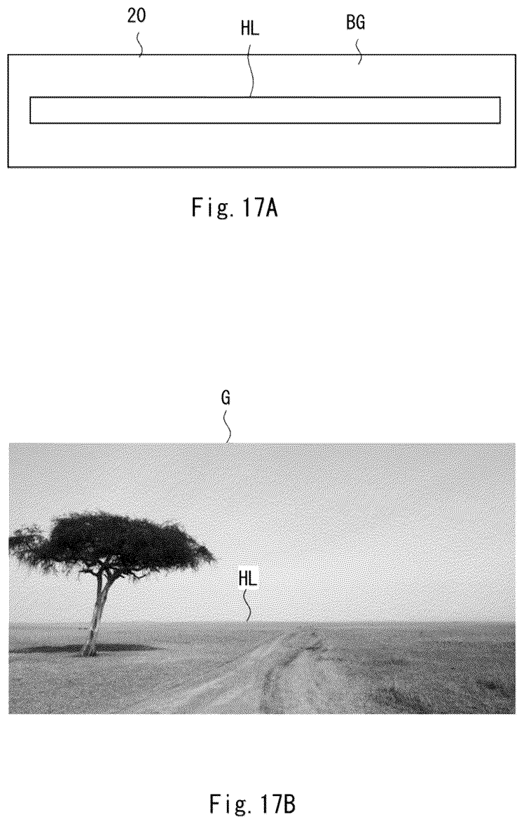

FIGS. 17A and 17B are variations of the display form of the horizontal line HL.

As shown in FIG. 17A, the horizontal line HL may be displayed on the film light source 20 together with the background color or background image BG. The background image BG may be a still image or a moving image.

Further, as shown in FIG. 17B, the image G including the horizontal line HL, for example, an image of a natural environment including the horizon corresponding to the horizontal line HL, may be displayed on the film light source 20. The image G may be a still image or a moving image. In this way, it can be expected to reduce motion sickness and to suppress nervous tension.

In the above embodiment, an example of using an automobile as the vehicle V has been described, but this is not limited thereto. In other words, the vehicle V may have any configuration as long as it is provided with the riding space in which individuals (one or more) ride. For example, it may be a vehicle such as a bus and other vehicles other than an automobile, a vehicle capable of automatic operation, a ship, a bus, a train, an airplane, or a spacecraft.

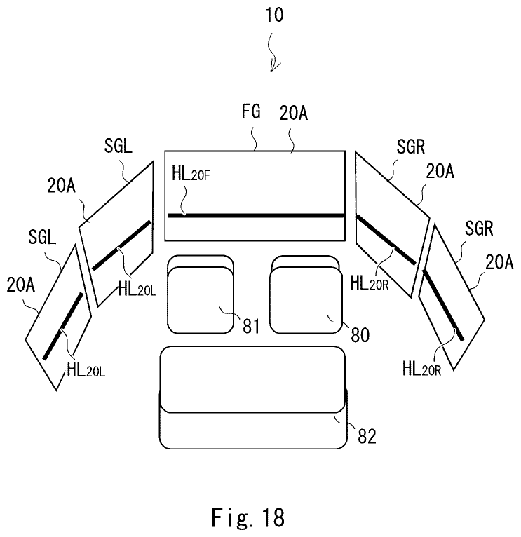

FIG. 18 shows a modification in which a liquid crystal panel 20A is used instead of the film light source 20.

For example, when a vehicle capable of automatic operation is used as the vehicle V, as shown in FIG. 18, a liquid crystal panel 20A may be provided in place of the film light source 20 so as to cover all of the windshield FG, side glasses SGL, SGR of the vehicle V constituting a wall surface surrounding the riding space.

By doing so, in addition to displaying the horizontal line HL, the lighting environment of the entire riding space can be controlled. For example, in addition to displaying the horizontal line HL (for example, in green) on the LCD panel 20A, a color that calms the nerves (for example, black) can be displayed as the background, thereby controlling the overall lighting environment of the riding space. This is expected to further reduce symptoms of motion sickness by controlling the functions of the sympathetic and parasympathetic nerves. Additionally, it can help suppress the excitation of the nerves.

In the above embodiment, an example of using the film light source 20 as the display device has been described, but this is not limited. The display device may have any configuration as long as it can display the horizontal line HL, such as a liquid crystal display installed in the riding space, a projector for projecting the horizontal line HL onto a screen (for example, a wall surrounding a riding space) installed in the riding space, or an aerial visual image drawing device for displaying (drawing) the horizontal line HL in the riding space itself. The representation medium of the horizontal line HL may be any medium provided in the riding space. In other words, there are no limitations on the surface and position of the medium for expressing the horizontal line HL.

Further, in the above embodiment, an example (see FIG. 10) in which the processing of step S12 and the following is executed when it is determined that a road condition satisfies a predetermined condition (step S11: YES) has been described, but this is not limited. For example, when an individual riding on the vehicle V turns on a horizontal line display switch (not shown) provided in the riding space, the processing of step S12 and the following may be executed. Similarly, an example (see FIG. 12) in which the processing of step S22 and the following is executed when it is determined that a road condition satisfies a predetermined condition (step S21: YES) has been described, but this is not limited. For example, when an individual riding on the vehicle V turns on a horizontal line display switch (not shown) provided in the riding space, the processing of step S22 and the following may be executed.

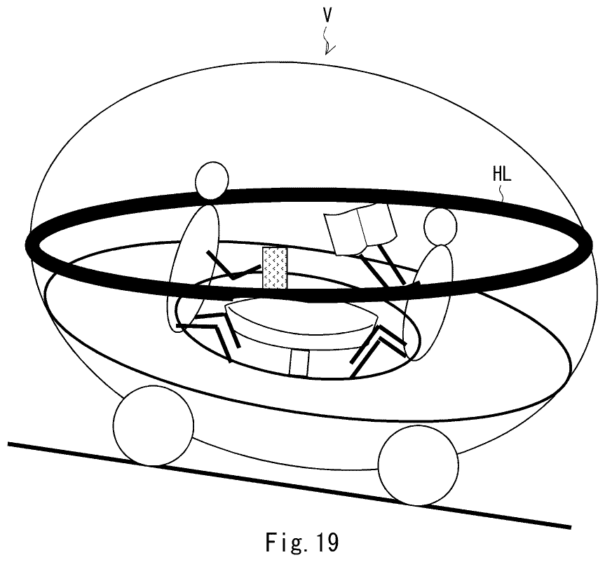

FIG. 19 shows a modification using a vehicle capable of automatic operation as the vehicle V.

In the above embodiment, an example of displaying the horizontal line HL on a part of the wall surface surrounding the riding space (windshield FG, side glasses SGL, SGR of the vehicle V constituting the wall) has been described (see FIG. 1), but this is not limited. The windshield refers to the glass or wall surface on the traveling direction side of the vehicle V, the side glass refers to the glass or wall surface on the vehicle width side (or the horizontal side perpendicular to the traveling direction) of the vehicle V, and the rear glass refers to the glass or wall surface on the opposite direction from the traveling direction side of the vehicle V. The windshield etc. may be made of a material other than glass, for example a plastic such as polycarbonate, or may be made of an opaque material, or even a colored material with transparency or an opaque colored material.

For example, as shown in FIG. 19, when a vehicle capable of automatic operation is used as the vehicle V, the horizontal line HL may be displayed on the entire circumference of the wall surrounding the riding space. FIG. 19 is a schematic diagram showing a vehicle V (a vehicle capable of automatic operation) traveling on a slope.

The numerical values described in the above-described embodiments are all illustrative, and appropriate numerical values different from the numerical values described in the above-described embodiments can be used as a matter of course.

The above-described embodiments are merely illustrative in all aspects. The present disclosure is not limitedly interpreted by the description of the above-described embodiments. The present disclosure can be implemented in other various forms without departing from the spirit or main features of the present disclosure.

This application is based on and claims the benefit of priority from Japanese Patent Application No. 2022-117739 filed on Jul. 25, 2022, the contents of which are hereby incorporated by reference.

### REFERENCE SIGNS LIST

10 . . . REDUCING APPARATUS
20 . . . FILM LIGHT SOURCE (DISPLAY DEVICE)
20A . . . LIQUID CRYSTAL PANEL
20F . . . FRONT FILM LIGHT SOURCE
20L . . . LEFT FILM LIGHT SOURCE
20R . . . RIGHT FILM LIGHT SOURCE
30 . . . ECU
31 . . . DETECTION RESULT ACQUISITION UNIT
32 . . . VEHICLE INCLINATION CALCULATION UNIT
33 . . . HORIZONTAL LINE DISPLAY UNIT
34 . . . ROAD CONDITION DETECTION UNIT
35 . . . HORIZONTAL LINE SETTING UNIT
40 . . . INERTIAL SENSOR
50 . . . AN IMAGING DEVICE
60 . . . NAVIGATION DEVICE
70 . . . INTERIOR LIGHT
71 . . . CONTROL CIRCUIT
Av . . . GRAVITY DIRECTION
AX_{H} . . . REFERENCE HORIZONTAL LINE
BG . . . BACKGROUND IMAGE
BP . . . BRIGHT SPOT
CP . . . REFERENCE CENTER POINT
FG . . . WINDSHIELD
G . . . IMAGE
HL . . . HORIZONTAL LINE
HL_{20F} . . . FRONT HORIZONTAL LINE
HL_{20L} . . . LEFT HORIZONTAL LINE
HL_{20R} . . . RIGHT HORIZONTAL LINE
HL_{S1}-HL_{S3} . . . HORIZONTAL LINE
PR1-PR3 . . . PROJECTORS
S1-S3 . . . SCREENS
SGL, SGR . . . SIDE GLASS
TS . . . SUBJECT
V . . . VEHICLE.
θ . . . PITCH ANGLE
φ . . . ROLL ANGLE

1. A motion sickness reducing apparatus, comprising:
a display device that is provided in a riding space of a vehicle having the riding space in which an individual rides and displays a horizontal line extending in a horizontal direction orthogonal to a gravity direction of the vehicle in the riding space regardless of a tilt of the vehicle.

2. The motion sickness reducing apparatus according to claim 1, further comprising:
a road condition detection unit for detecting a road condition where the vehicle is traveling; wherein
the display device displays the horizontal line when the road condition satisfies a predetermined condition.

3. The motion sickness reducing apparatus according to claim 2, wherein
the predetermined condition is a slope, a curved road, or a straight road traveling at a predetermined acceleration force or less.

4. The motion sickness reducing apparatus according to claim 1, wherein
the display device displays a bright spot moving along the horizontal line in a direction corresponding to an acceleration/deceleration of the vehicle together with the horizontal line.

5. The motion sickness reducing apparatus according to claim 1, wherein
the display device is provided on a windshield and a side glass constituting a wall surface surrounding the riding space.

6. The motion sickness reducing apparatus according to claim 5, wherein
the display device is further provided on a rear glass constituting the wall surface surrounding the riding space.

7. The motion sickness reducing apparatus according to claim 1, wherein
the display device is provided on a front dashboard and a side door provided in the riding space.

8. The motion sickness reducing apparatus according to claim 7, wherein
the display device is further provided on a rear dashboard provided in the riding space.

9. The motion sickness reducing apparatus according to claim 1, wherein
the display device is a film-shaped film light source.

10. The motion sickness reducing apparatus according to claim 9, wherein
the film light source is an organic EL or the film-shaped LED.

11. The motion sickness reducing apparatus according to claim 1, wherein
the display device is a liquid crystal display installed in the riding space, or a projector for projecting the horizontal line onto a screen installed in the riding space.

12. The motion sickness reducing apparatus according to claim 1, further comprising:
a horizontal line setting unit for setting at least one of a color and width of the horizontal line; wherein
the display device displays the horizontal line in the color, and width set.

13. The motion sickness reducing apparatus according to claim 1, wherein
the display device displays an image including the horizontal line.

14. The motion sickness reducing apparatus according to claim 1, wherein the display device is provided on at least a part of a wall surface surrounding four sides of the riding space.

15. A method for reducing motion sickness comprises displaying a horizontal line extending in a horizontal direction orthogonal to a gravity direction of a vehicle in a riding space of the vehicle provided with the riding space in which an individual rides regardless of a tilt of the vehicle.

16. The motion sickness reducing apparatus according to claim 4, wherein
the display device is provided on at least one side in the vehicle width direction within the riding space,
the bright spot starts from the rear end of the display device and moves, when the vehicle accelerates, and
the bright spot moves toward the end of the rear end of the display device, when the vehicle decelerates.

17. The motion sickness reducing apparatus according to claim 4, wherein
the display device is provided on the front side in the vehicle longitudinal direction within the riding space,
the bright spot moves toward the end of the center of the display device, when the vehicle accelerates, and
the bright spot starts from the center of the display device and moves, when the vehicle decelerates.

18. The motion sickness reducing apparatus according to claim 4, wherein
the display device is provided on at least one side in the vehicle width direction within the riding space,
the bright spot moves toward the rear end of the display device, when the vehicle accelerates, and
the bright spot starts from the rear end of the display device and moves, when the vehicle decelerates.

19. The motion sickness reducing apparatus according to claim 4, wherein
the display device is provided on the front side in the vehicle longitudinal direction within the riding space,
the bright spot starts from the center 1 of the display device and moves, when the vehicle accelerates, and
the bright spot moves toward the end of the center of the display device, when the vehicle decelerates.
